(19) United States
(12) Patent Application Publication
(10) Pub. No.: US 2015/0361034 A1
(43) Pub. Date: Dec. 17, 2015

(54) NEW PROCESS

(71) Applicant: Hoffmann-La Roche Inc., Nutley, NJ (US)

(72) Inventors: Gerard John Harnett, Ennis (IE); John Hayes, Ennis (IE); Reinhard Reents, Muenchenstein (CH); Dennis A. Smith, Ennis (IE); Andrew Walsh, Ennis (IE)

(73) Assignee: Hoffmann-La Roche Inc.

(21) Appl. No.: 14/831,389

(22) Filed: Aug. 20, 2015

Related U.S. Application Data
(63) Continuation of application No. 14/340,939, filed on Jul. 25, 2014, now abandoned.
Application No. 14/340,939 is a continuation of application No. 13/803,118, filed on Mar. 14, 2013, now abandoned.
Application No. 13/803,118 is a continuation of application No. 13/232,020, filed on Sep. 14, 2011, now abandoned.

(30) Foreign Application Priority Data
Sep. 16, 2010 (EP) 10177187.1

(51) Int. Cl. C07C 253/30; C07C 231/06; C07C 327/30; C07C 319/22; C07C 319/06; C07C 51/06; C07C 51/60

(52) U.S. Cl. CPC C07C 253/30; C07C 51/06; C07C 231/06; C07C 51/60; C07C 319/22; C07C 319/06; C07C 327/30; C07C 2101/14

(57) ABSTRACT

A process for the preparation of a compound of formula (I):

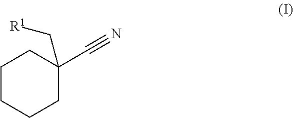

which is useful as an intermediate in the preparation of pharmaceutically active compounds.

### PRIORITY TO RELATED APPLICATION(S)

This application is a continuation application of U.S. application Ser. No. 13/803,118, filed on Mar. 14, 2013, which is a continuation of U.S. application Ser. No. 13/232,020, filed Sep. 14, 2011, which claims the benefit of European Patent Application No. 10177187.1, filed Sep. 16, 2010, which are hereby incorporated by reference in their entirety.

### SUMMARY OF THE INVENTION

The present invention relates to a process for the preparation of a cyclohexanecarboxylic acid derivative which is useful as an intermediate in the preparation of pharmaceutically active compounds.

### DETAILED DESCRIPTION OF THE INVENTION

In a first embodiment, the present invention provides a process for the preparation of a cyclohexanecarbonitrile derivative of formula (I):

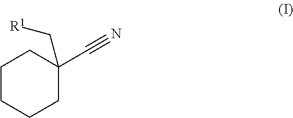

wherein R¹ is a (C₁-C₈)alkyl, preferably pent-3-yl, comprising adding a Grignard reagent, such as a (C₁-C₆)alkyl-magnesium-halide, phenyl-magnesium-halide, heteroaryl-magnesium-halide or a (C₃-C₆)cycloakyl-magnesium-halide to cyclohexanecarbonitrile of formula (II):

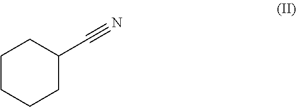

in the presence of an alkylating agent such as a 1-halo-CH₂R¹, preferably 1-halo-2-ethylbutane, or a sulfonate ester of R′CH₂—OH, preferably of 2-ethyl-1-butanol, wherein R¹ is as defined above.

In particular, the above mentioned coupling reaction is carried out in the presence of a secondary amine.

In particular, the above mentioned coupling reaction is followed by a mineral acid quenching, such as hydrofluoric acid, hydrochloric acid, boric acid, acetic acid, formic acid, nitric acid, phosphoric acid or sulfuric acid, most preferably by hydrochloric acid.

Contrary to expectation it was surprisingly found that adding the Grignard reagent to a mixture of the cyclohexanecarbonitrile and the alkylating agent, instead of first combining the Grignard reagent and the cyclohexanecarbonitrile before coupling with the alkylating agent, led to improved yields and a reduction in the formation of by-products. It is most surprising that the reaction is not complicated by the reaction between the Grignard reagent and the alkylating agent.

The compound of formula (I) may be used as intermediate in the synthesis of valuable pharmaceutical compounds. For example 1-(2-ethylbutyl)cyclohexanecarbonitrile may be used in the synthesis of the ones as described in EP 1,020,439 based on the intermediate process disclosed in WO 2009/121788.

Unless otherwise stated, the following terms used in the specification and claims have the meanings given below:

The term “halo” means fluoro, chloro, bromo or iodo. In particular embodiments, the halo is chloro or bromo.

The term “alkali metal” or “alkali” refers to lithium, sodium, potassium, rubidium or caesium. Preferable alkali metals are lithium and sodium. Of these, sodium is most preferred.

The term “(C₁-C₈)alkyl” refers to a branched or straight hydrocarbon chain of one to eight carbon atoms. Examples include methyl, ethyl, n-propyl, isopropyl, n-butyl, isobutyl, sec-butyl, t-butyl, pentyl, hexyl and heptyl. In particular embodiments, a (C₁-C₆)alkyl (i.e, a branched or straight hydrocarbon chain of one to six carbon atoms) is preferred.

The term “(C₁-C₆)alkoxy” means a moiety of the formula —OR^{ab}, wherein R^{ab} is a (C₁-C₆)alkyl moiety as defined herein. Examples of alkoxy moieties include, but are not limited to, methoxy, ethoxy, isopropoxy, and the like.

The term “(C₁-C₆)alkylene” means a linear saturated divalent hydrocarbon moiety of one to six carbon atoms or a branched saturated divalent hydrocarbon moiety of three to six carbon atoms. Examples include methylene, ethylene, 2,2-dimethylethylene, propylene, 2-methylpropylene, butylene, pentylene, and the like.

The term “halo-(C₁-C₈)alkyl” refers to an alkyl, as defined above, substituted with one or more halogen atoms. In particular embodiments, the halo-(C₁-C₈)alkyl is substituted with one to three halogen atoms. In other particular embodiments, the halo-(C₁-C₈)alkyl is chloro-(C₁-C₈)alkyl or fluoro-(C₁-C₈)alkyl.

The term “halo-(C₁-C₆)alkoxy” refers to an alkoxy, as defined above, substituted with one or more halogen atoms. In particular embodiments, the halo-(C₁-C₆)alkoxy is substituted with one to three halogen atoms. In other particular embodiments, the halo-(C₁-C₆)alkoxy is chloro-(C₁-C₆)alkoxy or fluoro-(C₁-C₆)alkoxy.

The term “(C₃-C₆)cycloalkyl” refers to a single saturated carbocyclic ring of three to six ring carbons. Examples include cyclopropyl, cyclobutyl, cyclopentyl and cyclohexyl. The (C₃-C₆)cycloalkyl may optionally be substituted with one or more substituents, preferably one, two or three, substituents; and is preferably selected from the group consisting of a (C₁-C₆)alkyl, hydroxy, a (C₁-C₆)alkoxy, a halo(C₁-C₆)alkyl, a halo(C₁-C₆)alkoxy, halo, amino, mono- or di(C₁-C₆)alkylamino, a hetero(C₁-C₆)alkyl, acyl, aryl and heteroaryl.

The term “secondary amine” refers to an amine of formula HNR²R³ wherein R² and R³ may be the same or different and are a (C₁-C₆)alkyl or (C₃-C₆)cycloalkyl, or R² and R³ taken together with the nitrogen atom to which they are attached, form a (C₄-C₈) heterocycloalkane optionally containing an additional heteroatom of O or N. Representative examples include, but are not limited to, piperidine, 4-methyl-piperidine, piperazine, pyrrolidine, morpholine, dimethylamine, diethylamine, diisopropylamine, dicyclohexylamine, ethylmethylamine, ethylpropylamine and methylpropylamine. Preferably, the secondary amine is chosen from diethylamine, diisopropylamine, dicyclohexylamine, ethylmethylamine, ethylpropylamine, methylpropylamine and morpholine. The more preferred secondary amine is diethylamine or diisopropylamine, and most preferred is diethylamine.

The term “(C₄-C₈)heterocycloalkane” refers to a saturated non-aromatic cyclic compound of 4 to 8 ring atoms in which one or two ring atoms are heteroatoms of N or O, and the heterocycloalkane is optionally substituted with one or more (C₁-C₃)alkyls, preferably one (C₁-C₃)alkyl.

The term “acyl” means a group of the formula —C(O)—R^{ag}, —C(O)—OR^{ag}, —C(O)—OC(O)R^{ag} or —C(O)—NR^{ag}R^{ah} wherein R^{ag} is hydrogen, (C₁-C₆)alkyl, halo(C₁-C₆)alkyl, heteroalkyl or amino as defined herein, and R^{ah} is hydrogen or (C₁-C₆)alkyl as defined herein.

The term “amino” means a group —NR^{ba}R^{bb} wherein R^{ba} and R^{bb} each independently is hydrogen or (C₁-C₆)alkyl.

The term “aryl” means a monovalent monocyclic or bicyclic aromatic hydrocarbon moiety which is optionally substituted with one or more substituents. In preferred embodiments, the aryl is optionally substituted with one, two or three substituents selected from the group consisting of a (C₁-C₆)alkyl, hydroxy, a (C₁-C₆)alkoxy, a halo(C₁-C₆)alkyl, a halo(C₁-C₆)alkoxy, halo, nitro, cyano, amino, mono- or di(C₁-C₆)alkylamino, methylenedioxy, ethylenedioxy, acyl, a hetero(C₁-C₆)alkyl, aryl, optionally substituted heteroaryl, optionally substituted arylalkyl, and optionally substituted heteroarylalkyl. A particularly preferred aryl substituent is halide. In more particular embodiments, the aryl is phenyl, 1-naphthyl, or 2-naphthyl, or the like, each of which can be substituted or unsubstituted.

The term “aralkyl” refers to a moiety of the formula —R^{bc}—R^{bd} where R^{bd} is aryl and R^{bc} is a (C₁-C₆)alkylene as defined herein.

The term “heteroaryl” means a monovalent monocyclic or bicyclic moiety of 5 to 12 ring atoms having at least one aromatic ring containing one, two, or three ring heteroatoms independently selected from the group consisting of N, O, and S with the remaining ring atoms being carbon, with the understanding that the attachment point of the heteroaryl moiety will be on an aromatic ring. In particular embodiments, the heteroaryl contains one, two, or three ring heteroatoms independently selected from the group consisting of N and O. In particular embodiments, the heteroaryl ring is optionally substituted independently with one or more substituents, preferably one, two or three substituents, each of which is independently selected from the group consisting of a (C₁-C₆)alkyl, halo(C₁-C₆)alkyl, hydroxy, a (C₁-C₆)alkoxy, halo, nitro and cyano. Examples of a heteroaryl include, but are not limited to, pyridyl, furanyl, thienyl, thiazolyl, isothiazolyl, triazolyl, imidazolyl, isoxazolyl, pyrrolyl, pyrazolyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, imidazo[1,2-a]-pyridinyl, imidazo[2,1-b]thiazolyl, and the derivatives thereof.

The term “nitrosylating agent” means a compound or composition comprising nitrosylsulfuric acid, sodium nitrite or a mixture thereof. Most preferably, the nitrosylating agent is nitrosylsulfuric acid.

The term “sulfonate ester” of R¹CH₂—OH″ or (R^{ca})(R^{cb})CH—OH refers to a substituted or an unsubstituted phenyl-sulfonate, an unsubstituted naphthalene-sulfonate or a (C₁-C₆)alkylsulfonate ester derivative of R¹CH₂—OH or (R^{ca})(R^{cb})CH—OH, respectively, wherein substituted phenyl and the (C₁-C₆)alkyl chain, R¹, R^{ca}, R^{cb} are as defined herein. Representative examples include, but are not limited to, benzenesulfonic acid 2-ethyl-butyl ester, 1-naphthalenesulfonic acid 2-ethyl-butyl ester, 2-naphthalenesulfonic acid 2-ethyl-butyl ester, toluene-4-sulfonic acid 2-ethyl-butyl ester, 4-nitro-benzenesulfonic acid 2-ethyl-butyl ester, 2,4,6-trimethyl-benzenesulfonic acid 2-ethyl-butyl ester, ethanesulfonic acid 2-ethyl-butyl ester, methanesulfonic acid 2-ethyl-butyl ester and butanesulfonic acid 2-ethyl-butyl ester.

The term “strong acid” refers to an acid that dissociates completely in an aqueous solution with a pH≦2. The strong acids include, but are not limited to: sulphuric acid (H₂SO₄), hydrohalogenic acid (i.e. HX″ wherein X″ is I, Br, Cl or F), nitric acid (HNO₃), phosphoric acid (H₃PO₄) and combinations thereof. Preferably, the strong acid is H₂SO₄ or hydrohalogenic acid, wherein X″ is Br or Cl. Most preferably, the strong acid is H₂SO₄. Preferably the concentration of H₂SO₄ in water is in the range of 75% to 90%, more preferably 78 to 83%, most preferably 82.5%.

The term “aqueous base” refers to a solution comprising a base and water. Numerous bases which readily dissolve in water are known in the art, such as NaOH, KOH, Ca(OH)₂, and Mg(OH)₂. In preferred embodiments the aqueous base is NaOH or KOH and/or the aqueous base has a pH of 12 to 14.

The term “substituent” refers to an atom or a group of atoms that replaces a hydrogen atom on a molecule. The term “substituted” denotes that a specified molecule bears one or more substituents.

The term “a compound of the formula” or “a compound of formula” or “compounds of the formula” or “compounds of formula” refers to any compound selected from the genus of compounds as defined by the formula.

In one embodiment the present invention provides a process comprising the synthetic steps represented in the following scheme 1:

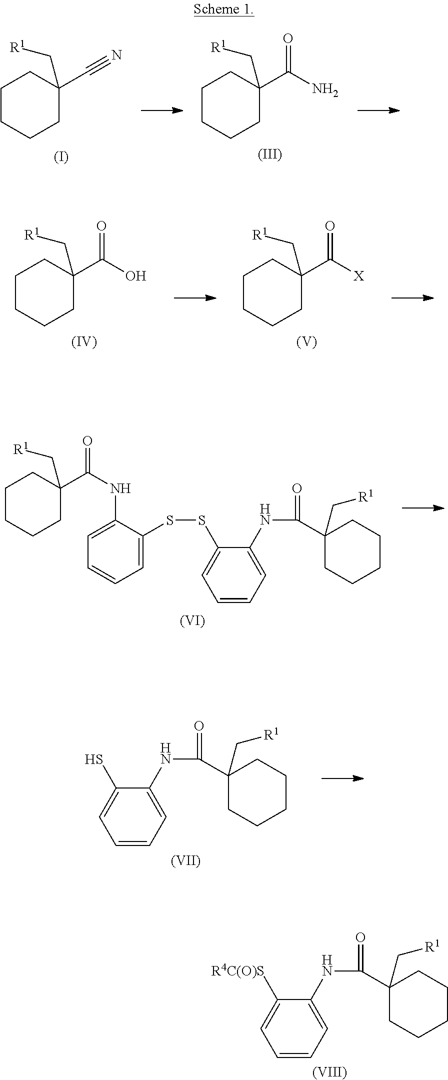

wherein X is I, Br, Cl or F, R¹ is as defined above and R⁴ is (C₁-C₈)alkyl. In particular, the process comprises hydrolysing a cyclohexanecarbonitrile derivative of formula (I) to obtain a cyclohexanecarboxylic acid amide derivative of formula (III) with for example H₂O in the presence of a strong acid, or with an aqueous base. The process further comprises reacting the said cyclohexanecarboxylic acid amide derivative with a nitrosylating agent, to obtain the compound of formula (IV). The process further comprises reacting a cyclohexanecarboxylic acid derivative of formula (IV) with a halogenating agent, such as PX₃, PX₅, SOX₂ or NCX, to obtain the acyl halide of formula (V). The halogenating step is preferably carried out in the presence of a tri-(C₁—O₅)alkylamine. Furthermore, the process comprises reacting acyl halide with bis(2-aminophenyl)disulfide to acylate the amino groups of the bis(2-aminophenyl)disulfide, reducing the amino-acylated disulfide product with a reducing agent such as triphenylphosphine, zinc or sodium borohydride to yield the thiol product, and acylating the thiol group in the thiol product with R⁴C(O)X′, wherein X′ is I, Br, Cl or F.

The additional steps may be performed, e.g., according to the procedures described in Shinkai et al., J. Med. Chem. 43:3566-3572 (2000), WO 2007/051714, WO2009121788.

Preferably the halogenating agent is chosen from thionyl chloride, phosphorus pentachloride, oxalyl chloride, phosphorus tribromide and cyanuric fluoride, most preferably thionyl chloride. The acyl halide of formula (V) wherein X is Cl is most preferred.

In the thiol acylation step, preferably the acylating agent is R⁴C(O)X′, wherein X′ is Cl. Most preferably R⁴ is isopropyl.

Unless otherwise stated, the organic solvent referred to herein comprises an ether like solvent (e.g. tetrahydrofuran, methyltetrahydrofuran, diisopropyl ether, t-butylmethyl ether or dibutyl ether, ethyl acetate, or butyl acetate), an alcohol solvent (e.g. methanol or ethanol), an aliphatic hydrocarbon solvent (e.g. hexane, heptane or pentane), a saturated alicyclic hydrocarbon solvent (e.g. cyclohexane or cyclopentane), or an aromatic solvent (e.g. toluene or t-butyl-benzene).

In a further embodiment, the present invention provides processes as described above wherein the nitrosylating agent is generated in situ; e.g. mixing H₂SO₄ and nitrous acid (HNO₂) or H₂SO₃/HNO₃ or N₂O₃/H₂SO₄ or HNO₃/SO₂ to obtain nitrosulfuric acic (NOHSO₄).

In another embodiment the invention provides a process for the preparation of a cyclohexanecarbonitrile derivative of formula (I):

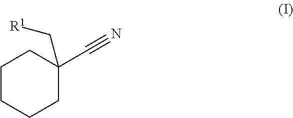

wherein R¹ is a (C₁-C₈)alkyl, preferably pent-3-yl, comprising adding a Grignard reagent, such as a (C₁-C₆)alkyl-magnesium-halide, phenyl-magnesium-halide, a heteroaryl-magnesium-halide or a (C₃-C₆)cycloalkyl-magnesium-halide to a solution or mixture comprising the cyclohexanecarbonitrile of formula (II), a secondary amine and an alkylating agent such as a 1-halo-CH₂R¹, preferably 1-halo-2-ethylbutane, or a sulfonate ester of R¹CH₂—OH, preferably of 2-ethyl-1-butanol, wherein R¹ is as defined above.

Within the processes defined above, preferably the halide of a Grignard reagent is chosen from chloride, bromide and iodide, more preferably chloride or bromide, and most preferably chloride.

The preferred alkyl of the Grignard reagent is a (C₁-C₃) alkyl, more preferably methyl. The most preferred Grignard reagent is methylmagnesiumchloride.

The preferred alkylating agent is 1-halo-2-ethylbutane, most preferably 1-bromo-2-ethylbutane.

Preferably, the alkylation is performed in the presence of a catalytic amount of a secondary amine, such as 0.01 to 0.5 equivalent of a secondary amine with respect to cyclohexanecarbonitrile, most preferably 0.05 eq. The dosing time of the Grignard reagent, is preferably 0.5 to 4 h, most preferably 1.5 h. This addition can be carried out at a temperature between 50 to 80° C., in particular between 60 to 75° C. After the addition of the Grignard reagent the reaction mixture can be stirred at reflux for a time, and in particular embodiments stirred for one hour.

A nonprotic organic solvent is the preferred solvent during the alkylation, such as tetrahydrofuran, alone or in combination with another nonprotic solvent, e.g. from the group of the apolar solvents hexane, heptane, methyl tetrahydrofurane, toluene and t-butyl-benzene, more preferably hexane, heptane, toluene and t-butyl-benzene. Most preferably the nonprotic solvent is tetrahydrofuran.

Preferably the hydrolysing agent of the cyclohexanecarbonitrile derivative of formula (I) is a strong acid. The most preferred strong acid is sulphuric acid. The hydrolysis step is either carried out by dosing a compound of formula (I) to sulphuric acid at a temperature of 80° C. to 120° C. or both a compound of formula (I) and sulphuric acid are heated as a mixture to a temperature of 80° C. to 120° C. More preferably the temperature in both modes of addition is 95 to 110° C., most preferably 105 to 110° C. 1.5 to 4 equivalents of sulphuric acid with respect to a compound of formula (I) is preferably used. More preferably 1.9 to 3.6 equivalents are used. Most preferably 2 equivalents are used. The hydrolysis is carried out with an excess of water, preferably 5 to 25 eq. of water with respect to the compound of formula (I), and more preferably 10 to 20 eq. Most preferably, 14 to 16 eq. of water is used with respect to the compound of formula (I).

For the hydrolysis of the amide of formula (III), preferably 1.1 to 1.4 equivalents of nitrosylsulfuric acid is used, most preferably 1.2 to 1.4 equivalents. Either nitrosylsulfuric acid is added first and followed by water or the water is first added and followed by the addition of nitrosylsulfuric acid. The second addition mode is preferred. Preferably, the dosing temperature is at 20 to 65° C., most preferably 60 to 65° C.

According to the present invention the “basic aqueous solution” for the extraction step (c) is preferably chosen from inorganic bases or organic bases, a mixture thereof, or from commonly known buffering solutions of suitable pH. The preferred inorganic base is an alkali base, such as alkali carbonate, alkali bicarbonate, alkali borate, alkali phosphate, alkali-hydroxide. A more preferred basic aqueous solution is chosen from a solution of potassium bicarbonate, sodium bicarbonate, potassium carbonate, sodium carbonate, sodium borate, sodium hydroxide, or a mixture thereof. The most preferred basic aqueous solution is a solution of sodium bicarbonate, sodium hydroxide or a mixture thereof.

In a further embodiment, the present invention provides a process for the preparation of [2-([[1-(2-ethylbutyl)-cyclohexyl]-carbonyl]amino)phenyl]2-methylpropanethioate comprising the formation of a compound of formula (I) obtained by any of the processes and conditions mentioned previously.

The starting materials and reagents, which do not have their synthetic route explicitly disclosed herein, are generally available from commercial sources or are readily prepared using methods well known to the person skilled in the art. For instance, a compound of formula (II) is commercially available or can be prepared by procedures known to the skilled person.

The methods of the present invention may be carried out as a semi-continuous or continuous processes, more preferably as a continuous processes.

The following examples are provided for the purpose of further illustration and are not intended to limit the scope of the claimed invention.

The following abbreviations and definitions are used: br (broad); BuLi (butyllithium); CDCl₃ (deuterated chloroform); eq. (equivalent); g (gram); GC (gas chromatography); h (hour); HCl (hydrochloric acid); H₂O (water); HPLC (High-Performance Liquid Chromatography); ISP (Isotopic Spin Population); KOH (Potassium Hydroxide); LDA (Lithium Diisopropylamide-); M (Molar); m (multiplet); MS (Mass Spectroscopy); mL (milliliter); NaOH (Sodium hydroxide); NMR (nuclear magnetic resonance); s (singlet); sec (second); t (triplet); THF (tetrahydrofuran);

### Example 1

### 1-(2-Ethyl-butyl)-cyclohexanecarbonitrile

Under argon 50.0 g cyclohexane carbonitrile (458 mmol), 1.68 g (2.39 mL) diethylamine (22.9 mmol, 0.05 eq.), 76.4 g (64.7 mL) 2-ethylbutyl bromide (463 mmol, 1.01 eq) and 101 g (114 mL) THF are added at 25° C. Then at a temperature of 70° C. using an infusion pump within 4 hours, 173 g methylmagnesiumchloride solution (3M) in THF (22.2% (m/m), 513 mmol, 1.12 eq.) are added. The reaction is stirred for 1 h at reflux temperature (73° C.). A conversion control sample shows <0.1% (red. area) cyclohexanecarbonitrile. After reaction completion the temperature of the reaction mixture is reduced to 66° C. 232 g (232 mL) water, 24.8 g (20.6 mL) HCl 37% (251 mmol, 0.55 eq), and 62 g (91.2 mL) heptane are charged under stirring at 25° C. The above hot reaction mixture (55° C.) is transferred from the reactor into the flask (25-60) within 15 minutes. The reactor is washed with 20 g (23 mL) THF and the wash solvent is also transferred into the Erlenmeyer flask. The biphasic mixture is stirred for 10 minutes. The two clear phases are separated and the lower aqueous phase is removed. The upper organic phase containing product is washed with 154 g water and concentrated at 50° C./<20 mbar. The residue is degassed at 50° C./<20 mbar. Obtained are 89.4 g 1-(2-ethyl-butyl)-cyclohexanecarbonitrile crude (assay: 93.8%, 434 mmol, yield: 94.2%) as a yellow to light brown oil. The product is transferred to a distillation flask. First the pressure in the distillation flask is reduced to 7 mbar, then 1-(2-ethyl-butyl)-cyclohexanecarbonitrile crude is heated slowly to 116° C. Collected are 6.56 g 1^{st} cut (1.75 g, assay: 78.8%, 2% yield) and 2^{nd} cut 4.81 g, assay: 93.9%, yield 5%) as a colorless to light yellow liquid at 109-116° C.) and then further cuts at 116-117° C.) to give 73.6 g 1-(2-ethyl-butyl)-cyclohexanecarbonitrile distilled (assay: 98.5%, yield 82%) as a colorless liquid. Discarded are 2.0 g of distillation residue as a brown liquid.

### Example 2

### 1-(2-Ethyl-butyl)-cyclohexanecarboxylic acid

Under argon 21.1 g (11.6 ml) sulfuric acid (96%) (207 mmol, 2.0 eq, contains 0.84 g water (47 mmol, 0.46 eq)) and 1.96 g water (109 mmol, 1.05 eq) is heated to 105° C. Tᵢ. 20.0 g of 1-(2-ethyl-butyl)-cyclohexanecarbonitrile (103 mmol, 1.0 eq) is added within 15 min at 105° C. Tᵢ and the reaction mixture is stirred for 2 h. A conversion control sample shows 1-(2-ethyl-butyl)-cyclohexanecarbonitrile <0.1%. The reaction mixture is cooled to 50° C. Tᵢ. Then 28.0 g of water (1.55 mol, 15 eq) is added within 5 minutes at 51° C. Tᵢ (exotherm). The reaction mixture temperature is adjusted to 61° C. Tᵢ and with vigorous stirring 36.2 g (19 mL) nitrosylsulfuric acid (40%) in sulfuric acid (114 mmol, 1.1 eq) is added constantly within 75 minutes at 60° C. Tᵢ. The reaction mixture is stirred for 45 minutes at 64° C. Tᵢ. A conversion control sample shows 0.2 norm % 1-(2-ethyl-butyl)-cyclohexanecarboxylic acid amide). To the biphasic mixture at 64° C. 20 g water is added and 13 g aq. HNO_{X} is evaporated at 131-137° C. and 1000 mbar. 20 g water is added and 20 g aq. HNO_{X} is evaporated at 131-137° C. and 1000 mbar. In the residue <50 ppm nitrite/nitrate are found. The reaction mixture is cooled to 20° C. 20.0 g (29.4 mL) Heptane are added and the biphasic mixture is stirred for 5 minutes. The lower aqueous phase is separated and discarded.

To the organic phase 20.0 g water is added and the biphasic mixture is stirred for 10 minutes. The lower aqueous phase is separated and discarded. 1-(2-ethyl-butyl)-cyclohexanecarboxylic acid in heptane is filtered using a paper filter and stored. The product containing the organic phase is distilled in a Dean-Stark-apparatus at 112° C. and 1000 mbar until no water can be removed in the water separator.

The organic phase is concentrated at 112° C. and 1000 mbar to a final volume of 40 mL (27.2 g) clear heptane phase. 10 g (14.7 mL) heptane are added. Obtained are 36.56 g of 1-(2-ethyl-butyl)-cyclohexanecarboxylic acid in heptane (91.3 mmol, assay 53.01%, contained weight: 19.38 g of 1-(2-Ethyl-butyl)-cyclohexanecarboxylic acid, yield 88.2%) as a light yellow to orange solution.

### Example 3

### 1-(2-Ethyl-butyl)-cyclohexanecarbonitrile

To a 1-litre jacketed flask fitted with a stirrer, thermometer, condenser and a pressure-equalised dropping funnel and purged with nitrogen were added cyclohexanecarbonitrile (21.8 g, 200 mmol), diethylamine (1.46 g, 20 mmol), 2-ethylbutybromide (33.3 g, 202 mmol) and tetrahydrofuran (44.0 g). The resulting clear solution was heated to 45° C. and stirred under a continuous stream of nitrogen. Methylmagnesium chloride in tetrahydrofuran (83 g of a 22% solution, 0.246 mmol) was added over one hour while maintaining the temperature of the reaction mixture between 45.3 and 61.4° C. The mixture was then refluxed between 67.4 and 70.2° C. for 75 minutes. Analysis of the reaction mixture by GLC showed 98.1% 1-(2-ethyl-butyl)-cyclohexanecarbonitrile, 0.9% ethylbutylbromide, 0.0% cyclohexanecarbonitrile and 0.2% acetylcyclohexane. The mixture was cooled to 48.7° C. then transferred over 25 minutes into a stirred mixture of deionised water (101 g), hydrochloric acid (37%, 10.8 g) and n-heptane (27 g) which had been precooled to 15° C. The temperature was kept between 15 and 60° C. during the addition. The reaction flask was rinsed with tetrahydrofuran (8.9 g) into the quenched mixture which was then cooled and agitated at between 15 and 30° C. for 20 minutes. After settling for 10 minutes the lower aqueous layer was split off. The remaining organic layer was washed with deionised water (68 g) before being concentrated under reduced pressure on the rotary evaporator at up to 60° C. until no further solvent distilled over. The product was further degassed under high vacuum at 80° C. to leave 38.3 g of pale yellow oil. The w/w assay of the product as determined by internal standard GLC was 95.8%, giving a contained yield of 1-(2-ethyl-butyl)-cyclohexanecarbonitrile of 36.7 g or 95.0% of theory. Area normalised assay by GLC showed 1-(2-ethyl-butyl)-cyclohexanecarbonitrile 99.1%, ethylbutyl bromide 0.2%, acetylcyclohexane 0.2% and others 0.5%.

### Example 4

### 1-(2-Ethyl-butyl)-cyclohexanecarbonitrile

To a 1-litre jacketed flask fitted with a stirrer, thermometer, condenser and pressure-equalised dropping funnel and purged with nitrogen were added cyclohexanecarbonitrile (21.8 g, 200 mmol), diethylamine (0.37 g, 20 mmol), 2-ethylbutyl bromide (33.3 g, 202 mmol) and tetrahydrofuran (44.0 g). The resulting clear solution was heated to 45 to 50° C. and stirred under a continuous stream of nitrogen. Methylmagnesium chloride in tetrahydrofuran (83 g of a 22% solution, 0.246 mmol) was added over 65 minutes while maintaining the temperature of the reaction mixture between 46.0 and 55.2° C. The mixture was then refluxed between 67.5 and 70.2° C. for 100 minutes. Analysis of the reaction mixture by gas liquid chromatography (GLC) showed 96.6% 1-(2-ethyl-butyl)-cyclohexanecarbonitrile, 2.0% ethylbutylbromide, 0.0% cyclohexanecarbonitrile and 0.9% acetylcyclohexane. The mixture was cooled to around 50° C. then transferred over 15 minutes into a stirred mixture of deionised water (101 g), hydrochloric acid (37%, 10.8 g) and n-heptane (27 g) which had been precooled to 15° C. The temperature was kept between 15 and 60° C. during the addition. The reaction flask was rinsed with tetrahydrofuran (8.9 g) into the biphasic mixture which was then cooled and agitated at between 15 and 30° C. for 20 minutes. After settling for 10 minutes the lower aqueous layer was split off. The remaining organic layer was washed with deionised water (68 g) before being concentrated under reduced pressure on the rotary evaporator at up to 50° C. until no further solvent was distilled over. The product was further degassed under high vacuum at 80° C. to leave 37.7 g of a pale yellow oil. The w/w assay of the product as determined by internal standard gas liquid chromatography (GLC) was 96.9%, giving a contained yield of 1-(2-ethyl-butyl)-cyclohexanecarbonitrile of 36.5 g or 94.6% of theory. An area normalised assay by GLC showed 1-(2-ethyl-butyl)-cyclohexanecarbonitrile 97.9%, ethylbutyl bromide 0.8%, acetylcyclohexane 1.1% and others at 0.2%.

All publications, patent applications, patents, and other references mentioned herein are incorporated by reference in their entirety.

1. A process for the preparation of a cyclohexanecarbonitrile derivative of formula (I): 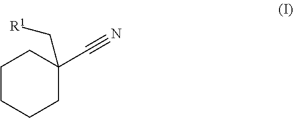
wherein R¹ is a (C₁-C₈)alkyl, comprising adding a Grignard reagent to the cyclohexanecarbonitrile of formula (II): 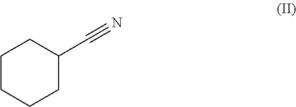
in the presence of an alkylating agent.

2. A process according to claim 1, wherein the coupling reaction is carried out in the presence of a secondary amine.

3. A process according to claim 2, wherein R¹ is pent-3-yl.

4. A process according claim 1 further comprising the preparation of a cyclohexanecarboxylic acid derivative of formula (IV): 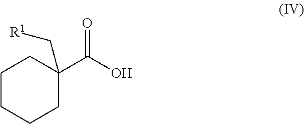
wherein R¹ is as defined in claim 1, comprising:
a) hydrolysing the cyclohexanecarbonitrile derivative of formula (I) in claim 1: 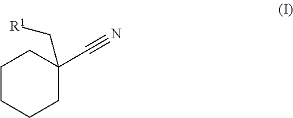
to obtain a cyclohexanecarboxylic acid amide derivative of formula (III): 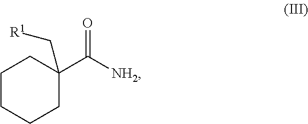
and b) further hydrolysing the compound of formula (III) to obtain the compound of formula (IV).

5. A process according to claim 4, further comprising the step of reacting the compound of formula (IV) as defined in claim 4 with a halogenating agent in the presence of a tri-(C₁-C₅)alkylamine, to obtain compound of formula (V): 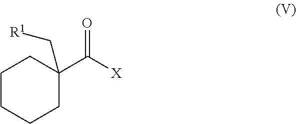
wherein R¹ is as defined in claim 4 and X is I, Br, Cl or F.

6. A process according to claim 5, further comprising the step of using the compound of formula (V) in claim 5, to acylate a compound of the formula VI′: 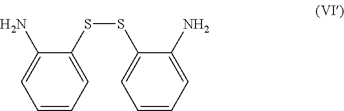 [SMILES: Nc1ccccc1SSc1ccccc1N]
to obtain a compound of formula VI: 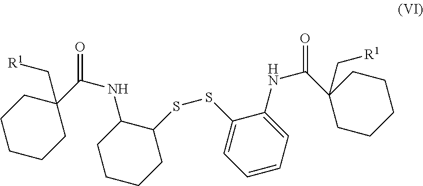
wherein R¹ is as defined in claim 5.

7. A process according to claim 6 further comprising the step of reducing the compound of formula VI as defined in claim 6 with a reducing agent to obtain a compound of formula VII: 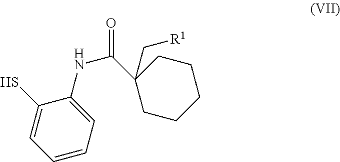
wherein R¹ is as defined in claim 6.

8. A process according to claim 7 further comprising the step of acylating the compound of formula VII as defined in claim 7 with R⁴C(O)X′, wherein X′ is I, Br, Cl or F, to obtain a compound of formula VIII: 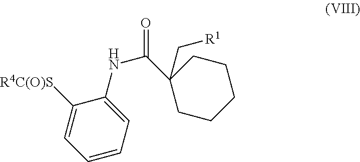
wherein R⁴ is a (C₁-C₈)alkyl and R¹ is as defined in claim 7.

9. A process according to claim 1, wherein the coupling reaction is followed by a mineral acid quenching with hydrofluoric acid, hydrochloric acid, boric acid, acetic acid, formic acid, nitric acid, phosphoric acid or sulfuric acid.

10. A process according to claim 1, wherein the coupling reaction is followed by a hydrochloric acid quenching.

11. A process according to claim 1, wherein a nonprotic solvent is present.

12. A process according to claim 11, wherein the nonprotic solvent is tetrahydrofuran.

13. A process according to claim 1, wherein the alkylating agent is 1-halo-CH₂R¹ or a sulfonate ester of R′CH₂—OH wherein R¹ is defined in claim 1.

14. A process according to claim 1, wherein the alkylating agent is 1-halo-2-ethylbutane.

15. A process according to claim 1, wherein the alkylating agent is 2-ethyl-1-butanol.

16. A process according to claim 1, wherein the alkylating agent is 1-bromo-2-ethylbutane.

17. A process according to claim 1, wherein the Grignard reagent is a (C₁-C₆)alkyl-magnesium-halide, phenyl-magnesium-halide, heteroaryl-magnesium-halide or a (C₃-C₆)cycloakyl-magnesium-halide.

18. A process according to claim 1, wherein the Grignard reagent is methylmagnesiumchloride.

19. A process according to claim 2, wherein the secondary amine is diethylamine or diisopropylamine.

20. A process according to claim 2, wherein the secondary amine is diethylamine.

21. A process according to claim 2, wherein the secondary amine is in a catalytic amount.

22. A process according to claim 2, wherein 0.01 to 0.5 equivalents of the secondary amine is used.

23. A process according to claim 2, wherein the process is continuous.

24. A process according to claim 8, wherein the compound of formula VIII is S-[2-([[1-(2-ethylbutyl)-cyclohexyl]-carbonyl]amino)phenyl]2-methylpropanethioate.
